(19) United States
(12) Patent Application Publication
(10) Pub. No.: US 2016/0074292 A1
(43) Pub. Date: Mar. 17, 2016

(54) MINERAL SALTS FOR REDUCING THE CONTENT OF CYSTEIC ACID IN KERATIN FIBRES

(71) Applicant: Henkel AG & Co. KGaA, Duesseldorf (DE)

(72) Inventors: Konstantin Goutsis, Juechen (DE); Gabriele Weser, Neuss (DE)

(21) Appl. No.: 14/946,373

(22) Filed: Nov. 19, 2015

Related U.S. Application Data
(63) Continuation of application No. PCT/EP2014/052010, filed on Feb. 3, 2014.

(30) Foreign Application Priority Data
May 22, 2013 (DE) 10 2013 209 498.1

(51) Int. Cl. A61K 8/19; A61K 8/23; A61K 8/22; A61Q 5/08; A61Q 5/10

(52) U.S. Cl. CPC A61K 8/19; A61Q 5/08; A61Q 5/10; A61K 8/22; A61K 8/23; A61K 2800/5922

(57) ABSTRACT

Cosmetic, non-therapeutic use of (a) sodium chloride in agents for oxidatively dyeing and/or oxidatively lightening keratinic fibers reduces the damage to the keratinic fibers resulting from use of the agents. Also, a ready-to-use agent for oxidatively dyeing and/or oxidatively lightening keratinic fibers includes (a) sodium chloride, (b) potassium chloride, (c) optionally at least one further salt from the group magnesium carbonate, magnesium hydrogen carbonate, magnesium sulfate, magnesium hydrogen sulfate, magnesium chloride, calcium carbonate, calcium hydrogen carbonate, calcium hydrogen sulfate, calcium sulfate, and/or calcium chloride, and (d) hydrogen peroxide.

### FIELD OF THE INVENTION

The present invention generally relates to the cosmetic use of particular mineral salts and the mixtures thereof in agents for oxidatively changing the color of keratinic fibers. By using the mineral salt mixtures, the aim is to reduce damage to keratinic fibers associated with use of the agents. A further subject matter of the present application relates to agents, including the mineral salt mixtures, for oxidatively changing the color of keratinic fibers. These agents are suited in particular for use in a method in which the agents are repeatedly applied to the keratinic fibers.

### BACKGROUND OF THE INVENTION

Changing the color of keratinic fibers, in particular hair, represents an important area of modern cosmetics. The appearance of the hair may thus be adapted to current fashion trends and to the person's individual preferences. Various options are known to those skilled in the art for changing the color of the hair. The hair color may be temporarily changed by using direct dyes. In the process, dyes which are already formed diffuse from the coloring agent into the hair fiber. Although coloration using direct dyes involves little damage to the hair, it is disadvantageous that the colorings obtained with direct dyes are not very durable and wash out quickly.

If the user desires a long-lasting color result or a shade that is lighter than his/her original hair color, oxidative color-changing agents are customarily used. For long-lasting, intense colorations having appropriate fastness properties, so-called oxidation dyes are used. Such coloring agents customarily include oxidation dye precursors, so-called developer components and coupler components, which form the actual dyes with one another under the influence of oxidizing agents. Oxidation dyes are characterized by excellent, long-lasting color results.

As oxidizing agent, as a rule hydrogen peroxide is used, which not only initiates the dye formation process but also oxidatively destroys the hair's own melanin pigments. Lightening of the hair is thus achieved at the same time. The quantity of hydrogen peroxide used is usually selected depending on the desired lightening effect. Oxidative coloring agents generally include between 3 and 12% by weight hydrogen peroxide.

Strictly lightening or bleaching of hair often takes place using oxidizing agents without the addition of oxidation dye precursors. For a medium bleaching effect, use of hydrogen peroxide alone as oxidizing agent is sufficient; for achieving a more intense bleaching effect, a mixture of hydrogen peroxide and peroxydisulfate salts is customarily used.

However, the lightening is also necessarily accompanied by damage to the hair, since not only the melanin pigments, but also the other structural components of the hair are oxidatively damaged. The melanin pigments are present in the form of granules in the cortex cells, i.e., in the interior of the hair. With the oxidative destruction of the melanin granules, hydrogen peroxide not only damages the outer scale layer of the hair (cuticle), but also oxidatively degrades other structural components present in the interior of the hair fiber.

The most important components of hair fibers are amino acids, which are linked by amide bonds in the protein molecules of the keratin fibers to form macromolecules. Due to the influence of hydrogen peroxide, in addition to melanin degradation, chemical degradation reactions also take place at these polypeptide chains, for example at the cystine bridges, the amide and amino groups, and at the hydrogen bridges and salt bonds of the peptide structure. These degradation reactions result in significant changes in the original mechanical properties of the hair.

Hair keratins are proteins having an extraordinarily high proportion (up to 10% by weight) of cystine.

Two units of the amino acid cysteine, which are present in different peptide strands, are covalently linked by the cystine via a disulfide structural unit. The cystines included in the hair are of major importance for the mechanical stability of the hair.

When oxidative color-changing agents are used, only a portion of the cystine bridges present in the keratin fiber are oxidatively degraded. In the process, the disulfide groups of the cystine are oxidatively cleaved and converted to sulfonic acid units. The cystine linking the various peptide strands is thus oxidized to form two cysteic acid units which are discrete, i.e., no longer bridged.

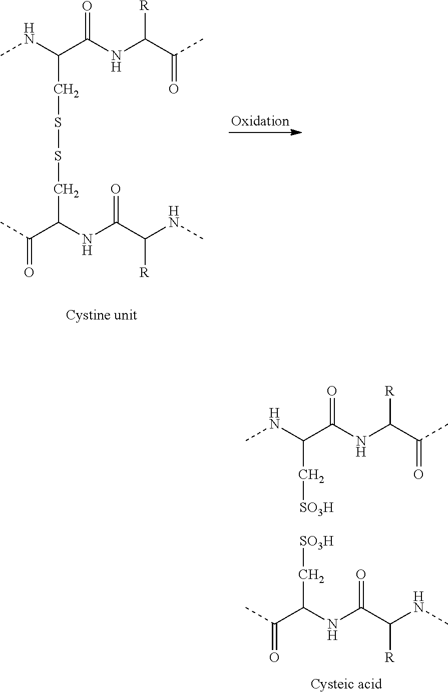

Due to these cleavage reactions, the number of linkage sites among the peptide strands decreases, and the mechanical stability of the keratin fiber is significantly impaired. The more often the oxidative color-changing agents are used and the higher the hydrogen peroxide content in the agents (either alone or in combination with other oxidizing agents), the greater the loss of stability of the peptide backbone. This is manifested in particular by reduced resistance and decreased tear strength of the keratin fibers. In addition, their extensibility, in particular in the wet state, is greatly increased. All of these accompanying factors of oxidative color-changing agents are highly undesirable to the user.

Those skilled in the art are familiar with various care and conditioner active substances whose use in oxidative color-changing agents is described. These care active substances are typically cationic surfactants or cationic polymers, which due to their positive charge(s) accumulate on the negatively charged surface (i.e., the negatively charged cuticle) of the hair, thus smoothing or conditioning the surface of the fibers. An improvement in the combability, for example, may be achieved via this mechanism. However, these are solely surface effects, since these organic compounds, due to their relatively high molar mass and their large molecular structure, are not able to diffuse into the interior of the hair fiber and directly prevent oxidative degeneration of the peptidic fiber backbone at that location. Even for the oils and polymeric silicone compounds which are likewise often used for conditioning, these are analogous surface effects.

EP 2 417 964 A1 describes a method for reducing the cysteic acid content in the chemical treatment of keratin fibers, in which diamides based on organic diacids are used. These diacids may include up to 20 C atoms, and thus likewise represent relatively large organic compounds.

A major disadvantage of these organic conditioner active substances known from the literature is their accumulation on the surface of the keratin fibers, which becomes noticeable after multiple applications of the agents. With each application, a portion of the care substances deposits on the hair surface. When there is an appropriately high affinity of the compounds for the hair surface (for example, due to their positive charge or due to their hydrophobicity), an accumulation effect may result, in particular when the consumer repeats the treatment within very short time intervals, which may lead to problem hair, less fullness of the hair, a greasy feeling on the hair (in particular with oils and silicones), or increased static charge, i.e., “fly-away hair” (in particular for cationic surfactants and cationic polymers). The result is “overtreatment” of the hair and an unpleasant feel of the hair.

Oxidative color-changing agents which have a direct positive effect on the cysteic acid content of the hair without at the same time having these disadvantages are thus far not known from the prior art.

It is therefore desirable to provide oxidative color-changing agents having excellent dyeing and lightening power and reduced damage to the hair. The aim is that keratinic fibers which have been treated with such oxidative color-changing agents are characterized by a reduced cysteic acid content and improved mechanical stability, greater tear strength, and reduced extensibility. The aim is to reduce the cysteic acid content of the keratinic fibers, in particular even after multiple applications of the oxidative color-changing agents.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

It has now surprisingly been found that keratinic fibers have a reduced cysteic acid content when they are treated with oxidative color-changing agents which include sodium chloride, either alone or in particular in a mixture with other mineral salts, as inorganic mineral salt. The agents including these mineral salt mixtures do not have the above-described disadvantages; i.e., the agents may be applied multiple times in succession, even within short time intervals, without the active substance accumulating on the surface of the keratin fibers.

The present invention includes the cosmetic, non-therapeutic use of sodium chloride in agents for oxidatively dyeing and/or oxidatively lightening keratinic fibers while reducing the damage to the keratinic fibers resulting from use of the agents.

The present invention also includes a ready-to-use agent for oxidatively dyeing and/or oxidatively lightening keratinic fibers, which includes sodium chloride; potassium chloride; optionally at least one further salt from the group magnesium carbonate, magnesium hydrogen carbonate, magnesium sulfate, magnesium hydrogen sulfate, magnesium chloride, calcium carbonate, calcium hydrogen carbonate, calcium hydrogen sulfate, calcium sulfate, and/or calcium chloride; and hydrogen peroxide.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject matter of the present invention relates to the cosmetic, non-therapeutic use of

(a) sodium chloride
in agents for oxidatively dyeing and/or oxidatively lightening keratinic fibers while reducing the damage to the keratinic fibers resulting from use of the agents.

Keratinic fibers, keratin-containing fibers, or keratin fibers are understood to mean fur, wool, feathers, and in particular human hair. Although the agents according to the invention are primarily suited for lightening and dyeing keratin fibers, use in other fields is also possible in principle.

Within the meaning of the present invention, the reduction of damage to the keratinic fibers resulting from use of the agents is understood to mean the reduction of the structural damage to the protein backbone responsible for the mechanical strength of the fiber.

A preferred embodiment, therefore, is the cosmetic, non-therapeutic use of

(a) sodium chloride
in agents for oxidatively dyeing and/or oxidatively lightening keratinic fibers while reducing the structural damage to the keratinic fibers resulting from use of the agents.

A particularly preferred embodiment is the cosmetic, non-therapeutic use of

(a) sodium chloride
in agents for oxidatively dyeing and/or oxidatively lightening keratinic fibers while reducing the cysteic acid content of the keratinic fibers treated with these agents.

The reduced cysteic acid content of the oxidatively treated keratinic fibers has direct positive effects on the mechanical stability and the tear strength of the keratinic fibers.

Another preferred embodiment is therefore the cosmetic, non-therapeutic use of

(a) sodium chloride
in agents for oxidatively dyeing and/or oxidatively lightening keratinic fibers while improving the mechanical stability of the keratinic fibers treated with these agents.

Another preferred embodiment is also the cosmetic, non-therapeutic use of

(a) sodium chloride
in agents for oxidatively dyeing and/or oxidatively lightening keratinic fibers while improving the tear strength of the keratinic fibers treated with these agents.

Sodium chloride (a) is the compound NaCl, also referred to as table salt or rock salt. Sodium chloride of various origins may be used. The sodium chloride may be used either in its crystalline form or in the form of a brine (aqueous NaCl solution). The use of sodium chloride originating from seawater, or seawater itself, is also possible. Also preferred is the use of sodium chloride mixed with other mineral salts, which is the case, for example, for water from mineral springs.

Sodium chloride from the La Toja mineral spring is very particularly preferably used, either in the form of the water from the spring or in the form of the residue resulting from evaporation of the La Toja spring water.

It has been possible to effectively reduce the cysteic acid content in particular for keratinic fibers which have been treated with oxidative color-changing agents including sodium chloride mixed with one or more further mineral salts. Good measuring results have been obtained in particular when the oxidative color-changing agents include a mixture of sodium chloride and potassium chloride.

A very particularly preferred use is therefore characterized in that the agents additionally include

(b) potassium chloride.

A particularly preferred embodiment is therefore the cosmetic, non-therapeutic use of

(a) sodium chloride and
(b) potassium chloride
in agents for oxidatively dyeing and/or oxidatively lightening keratinic fibers while reducing the damage to the keratinic fibers resulting from use of the agents.

Another particularly preferred embodiment is the cosmetic, non-therapeutic use of

(a) sodium chloride and
(b) potassium chloride
in agents for oxidatively dyeing and/or oxidatively lightening keratinic fibers for reducing the structural damage to the keratinic fibers resulting from use of the agents.

Another particularly preferred embodiment is the cosmetic, non-therapeutic use of

(a) sodium chloride and
(b) potassium chloride
in agents for oxidatively dyeing and/or oxidatively lightening keratinic fibers while reducing the cysteic acid content of the keratinic fibers treated with these agents.

Another preferred embodiment is therefore the cosmetic, non-therapeutic use of

(a) sodium chloride and
(b) potassium chloride
in agents for oxidatively dyeing and/or oxidatively lightening keratinic fibers while improving the mechanical stability of the keratinic fibers treated with these agents.

Another preferred embodiment is also the cosmetic, non-therapeutic use of

(a) sodium chloride and
(b) potassium chloride
in agents for oxidatively dyeing and/or oxidatively lightening keratinic fibers while improving the tear strength of the keratinic fibers treated with these agents.

Oxidative color-changing agents which include sodium chloride (a) in specific quantity ranges have been found to be particularly effective for reducing oxidative damage to the protein backbone of the fiber. Use of the corresponding agents is therefore particularly preferred.

One particularly preferred use is therefore further characterized in that the agents include sodium chloride (a) in a quantity of 0.05 to 3.5% by weight, preferably 0.1 to 2.5% by weight, more preferably 0.2 to 1.5% by weight, and particularly preferably 0.3 to 0.8% by weight, based on the total weight of the ready-to-use agents.

Potassium chloride (b) is preferably used in the oxidative color-changing agents in a quantity of 0.01 to 1.0% by weight, preferably 0.02 to 0.5% by weight, more preferably 0.03 to 0.3% by weight, and particularly preferably 0.04 to 0.1% by weight, based on the total weight of the ready-to-use agents.

In another particularly preferred embodiment, a use according to the invention is therefore characterized in that the agents include potassium chloride (b) in a quantity of 0.01 to 1.0% by weight, preferably 0.02 to 0.5% by weight, more preferably 0.03 to 0.3% by weight, and particularly preferably 0.04 to 0.1% by weight, based on the total weight of the ready-to-use agents.

Very particularly preferred is the cosmetic, non-therapeutic use of

(a) sodium chloride and
(b) potassium chloride
in agents for oxidatively dyeing and/or oxidatively lightening keratinic fibers while reducing the damage to the keratinic fibers resulting from use of the agents, wherein the agents include

sodium chloride (a) in a quantity of 0.05 to 3.5% by weight, preferably 0.1 to 2.5% by weight, more preferably 0.2 to 1.5% by weight, and particularly preferably 0.3 to 0.8% by weight, based on the total weight of the ready-to-use agents, and

potassium chloride (b) in a quantity of 0.01 to 1.0% by weight, preferably 0.02 to 0.5% by weight, more preferably 0.03 to 0.3% by weight, and particularly preferably 0.04 to 0.1% by weight, based on the total weight of the ready-to-use agents.

With regard to reducing the damage to hair, it has proven to be particularly advantageous when sodium chloride (a) and potassium chloride (b) are used as a mixture in specific weight ratios in the oxidative color-changing agents. In this regard, the use of an excess of sodium chloride is particularly advantageous.

Very particularly preferred, therefore, is a use which is characterized in that the agents include sodium chloride (a) and potassium chloride (b) in a weight ratio (a)/(b) of 20:1 to 1:1, preferably 18:1 to 3:1, more preferably 16:1 to 6:1, and particularly preferably 14:1 to 9:1, based on the total weight of the ready-to-use agents.

Accordingly, likewise very particularly preferred is the cosmetic, non-therapeutic use of

(a) sodium chloride and
(b) potassium chloride
in agents for oxidatively dyeing and/or oxidatively lightening keratinic fibers while reducing the damage to the keratinic fibers resulting from use of the agents, wherein the agents include

sodium chloride (a) and potassium chloride (b) in a weight ratio (a)/(b) of 20:1 to 1:1, preferably 18:1 to 3:1, more preferably 16:1 to 6:1, and particularly preferably 14:1 to 9:1, based on the total weight of the ready-to-use agents.

In addition to sodium chloride (a) and optionally potassium chloride (b), the oxidative color-changing agents may also include further inorganic salts or the mixtures thereof. In this regard, it has been found that adding one or more salts from the group magnesium carbonate, magnesium hydrogen carbonate, magnesium sulfate, magnesium hydrogen sulfate, magnesium chloride, calcium carbonate, calcium hydrogen carbonate, calcium hydrogen sulfate, calcium sulfate, and/or calcium chloride further increases the cysteic acid content and the tear strength of the keratinic fibers.

Therefore, a use is also particularly preferred which is further characterized in that the agents additionally include at least one further salt (c) from the group magnesium carbonate, magnesium hydrogen carbonate, magnesium sulfate, magnesium hydrogen sulfate, magnesium chloride, calcium carbonate, calcium hydrogen carbonate, calcium hydrogen sulfate, calcium sulfate, and/or calcium chloride.

In other words, the cosmetic, non-therapeutic use of

(a) sodium chloride
(b) potassium chloride and
(c) at least one further salt from the group magnesium carbonate, magnesium hydrogen carbonate, magnesium sulfate, magnesium hydrogen sulfate, magnesium chloride, calcium carbonate, calcium hydrogen carbonate, calcium hydrogen sulfate, calcium sulfate, and/or calcium chloride
is therefore particularly preferred in agents for oxidatively dyeing and/or oxidatively lightening keratinic fibers while reducing the damage to the keratinic fibers resulting from use of the agents.

Likewise particularly preferred is the use of the first subject matter of the invention, which is characterized in that

the agents include at least one combination of two different salts of group (c), which is selected from calcium chloride/magnesium carbonate, calcium chloride/magnesium hydrogen carbonate, calcium chloride/magnesium sulfate, calcium chloride/magnesium hydrogen sulfate, calcium chloride/magnesium chloride, calcium chloride/calcium carbonate, calcium chloride/calcium hydrogen carbonate, calcium chloride/calcium hydrogen sulfate, and/or calcium chloride/calcium sulfate.

In other words, the cosmetic, non-therapeutic use of

(a) sodium chloride
(b) potassium chloride and
(c) at least one combination of two different salts from the group calcium chloride/magnesium carbonate, calcium chloride/magnesium hydrogen carbonate, calcium chloride/magnesium sulfate, calcium chloride/magnesium hydrogen sulfate, calcium chloride/magnesium chloride, calcium chloride/calcium carbonate, calcium chloride/calcium hydrogen carbonate, calcium chloride/calcium hydrogen sulfate, and/or calcium chloride/calcium sulfate is therefore particularly preferred in agents for oxidatively dyeing and/or oxidatively lightening keratinic fibers while reducing the damage to the keratinic fibers resulting from use of the agents.

Agents for oxidatively dyeing or oxidatively lightening keratinic fibers include at least one oxidizing agent for initiating the dyeing process and for oxidatively destroying the hair's own melanin pigments (i.e., for producing the lightening effect). This oxidizing agent is typically hydrogen peroxide. In the course of the studies leading to the present invention, it has been experimentally demonstrated that sodium chloride (a), either alone or in combination with the above-described further mineral salts, is very well suited for reducing structural damage to the keratin fibers caused by hydrogen peroxide.

Another preferred use is therefore characterized in that the agents include hydrogen peroxide as oxidizing agent (d).

In other words, the cosmetic, non-therapeutic use of

(a) sodium chloride
is therefore particularly preferred in agents for oxidatively dyeing and/or oxidatively lightening keratinic fibers for reducing the damage to the keratinic fibers resulting from use of the agents, the agents including hydrogen peroxide as oxidizing agent (d).

It has been found that sodium chloride (a) minimizes the harmful effects of hydrogen peroxide (d) on the peptide structure of the hair, in particular when sodium chloride and hydrogen peroxide are used in specific quantity ratios relative to one another. To significantly reduce the cysteic acid content of the keratin fibers, it is advantageous to use at least 1 part by weight sodium chloride (a) to 10 parts by weight hydrogen peroxide (d).

One preferred use is therefore further characterized in that the agents include sodium chloride (a) and hydrogen peroxide (d) in a weight ratio (a)/(d) of 2:1 to 1:10, preferably 1:1 to 1:8, more preferably 1:2 to 1:8, and particularly preferably 1:3 to 1:6, based on the total weight of the ready-to-use agents.

In other words, the cosmetic, non-therapeutic use of

(a) sodium chloride
is therefore particularly preferred in agents for oxidatively dyeing and/or oxidatively lightening keratinic fibers while reducing the damage to the keratinic fibers resulting from use of the agents, the agents including sodium chloride (a) and hydrogen peroxide (d) in a weight ratio (a)/(d) of 2:1 to 1:10, preferably 1:1 to 1:8, more preferably 1:2 to 1:8, and particularly preferably 1:3 to 1:6, based on the total weight of the ready-to-use agents.

The concept of oxidative color changing encompasses the oxidative dyeing as well as the oxidative lightening of keratinic fibers.

The agents for oxidatively dyeing and/or oxidatively lightening keratinic fibers include the above-described ingredients essential to the invention in a cosmetic carrier. The cosmetic carrier is preferably aqueous, alcoholic, or aqueous-alcoholic. For example, creams, emulsions, gels, or also surfactant-containing foaming solutions, such as foam aerosols or foam nonaerosol applications, are suitable for application to the hair. Within the meaning of the present invention, aqueous-alcoholic carriers are understood to mean water-containing compositions including 3 to 70% by weight of a C₁-C₄ alcohol, in particular ethanol or isopropanol.

The oxidizing agent included in the oxidative color-changing agents is preferably hydrogen peroxide and/or one of its solid addition products with organic or inorganic compounds.

In one preferred embodiment, hydrogen peroxide itself is used as the aqueous solution. The concentration of a hydrogen peroxide solution in the agent according to the invention is determined on the one hand by regulatory requirements, and on the other hand by the desired effect; 6 to 12% by weight solutions in water are preferably used. Ready-to-use agents of the first subject matter of the invention which are preferred according to the invention are characterized in that, based on the total weight of the ready-to-use agent, they include 0.5 to 20% by weight, preferably 1 to 12.5% by weight, particularly preferably 2.5 to 10% by weight, and in particular 3 to 6% by weight, of hydrogen peroxide, in each case based on the total weight of the agent.

In addition to the oxidizing agent, oxidative coloring agents include at least one oxidation dye precursor, preferably at least one oxidation dye precursor of the developer type and at least one oxidation dye precursor of the coupler type. Particularly suitable oxidation dye precursors of the developer type are selected from at least one compound from the group comprising p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diaminopropan-2-ol, bis-(2-hydroxy-5-aminophenyl)methane, 1,3-bis-(2.5-diaminophenoxy)propan-2-ol, N,N′-bis-(4-aminophenyl)-1,4-diazacycloheptane, 1,10-bis-(2.5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo[1,2-a]pyrazol-1-one, and the physiologically acceptable salts thereof.

Particularly suitable oxidation dye precursors of the coupler type are selected from the group comprising 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or mixtures of these compounds or the physiologically acceptable salts thereof.

In addition, the oxidative coloring agents may likewise include at least one direct dye from the group of anionic, nonionic, and/or cationic dyes.

The oxidation dye precursors, i.e., developer components and coupler components, and the optionally additionally included direct dyes are preferably used in each case in a quantity of 0.0001 to 5.0% by weight, preferably 0.001 to 3.5% by weight, in each case based on the total weight of the ready-to-use agent. Developer components and coupler components are generally used in approximately molar quantities relative to one another. Even if the molar use has proven to be practical, a certain excess of individual oxidation dye precursors is not disadvantageous, so that developer components and coupler components may be in a mole ratio of 1:0.5 to 1:3, in particular 1:1 to 1:2.

Agents for oxidatively lightening (i.e., bleaching) keratinic fibers may likewise include oxidation dye precursors in fairly small quantities for shading. In one preferred embodiment, however, the agents are free of oxidation dye precursors. To achieve a more intense lightening effect, oxidative lightening agents may include hydrogen peroxide in combination with one or more further oxidizing agents, in particular including peroxo salts.

Suitable peroxo salts are inorganic peroxo compounds preferably selected from the group comprising ammonium peroxodisulfate, alkali metal peroxodisulfates, ammonium peroxomonosulfate, alkali metal peroxomonosulfates, alkali metal peroxodiphosphates, and alkaline earth metal peroxides. Peroxodisulfates, in particular ammonium peroxodisulfate, potassium peroxodisulfate, and sodium peroxodisulfate, are particularly preferred.

The persulfates are in each case included in the agents for oxidatively lightening keratinic fibers in a quantity of 0.5 to 20% by weight, preferably 1 to 12.5% by weight, particularly preferably 2.5 to 10% by weight, and in particular 3 to 6% by weight, based on the total weight of the ready-to-use agent.

As already described above, oxidative color-changing agents which include sodium chloride (a) in combination with potassium chloride (b) are particularly suitable for low-damage dyeing and lightening of keratinic fibers. The reduced damage is manifested primarily in that the fibers treated (in particular multiple times) with the agents have a reduced cysteic acid content.

A second subject matter of the present invention therefore relates to a ready-to-use agent for oxidatively dyeing and/or oxidatively lightening keratinic fibers, including

(a) sodium chloride,
(b) potassium chloride, and
(d) hydrogen peroxide.

The cysteic acid content of the treated keratin fibers may be reduced even further by using at least one further mineral salt from a specific group.

One preferred embodiment is therefore a ready-to-use agent for oxidatively dyeing and/or oxidatively lightening keratinic fibers, including

(a) sodium chloride,
(b) potassium chloride,
(c) optionally at least one further salt from the group magnesium carbonate, magnesium hydrogen carbonate, magnesium sulfate, magnesium hydrogen sulfate, magnesium chloride, calcium carbonate, calcium hydrogen carbonate, calcium hydrogen sulfate, calcium sulfate, and/or calcium chloride, and
(d) hydrogen peroxide.

One particularly preferred embodiment is a ready-to-use agent for oxidatively dyeing and/or oxidatively lightening keratinic fibers, including

(a) sodium chloride,
(b) potassium chloride,
(c) at least one further salt from the group magnesium carbonate, magnesium hydrogen carbonate, magnesium sulfate, magnesium hydrogen sulfate, magnesium chloride, calcium carbonate, calcium hydrogen carbonate, calcium hydrogen sulfate, calcium sulfate, and/or calcium chloride, and
(d) hydrogen peroxide.

Oxidative color-changing agents are generally provided in the form of a two- or multicomponent agent. The first component (I) includes an alkalizing agent, and, if shading or coloration is to be carried out in addition to lightening, the oxidation dye precursors. The second component (II) includes the oxidizing agent or the oxidizing agent mixture. The ready-to-use agent is prepared by the user immediately before application by mixing the two components (I) and (II).

Component (I) including the alkalizing agent (and optionally the oxidation dye precursors), and component (II) including the oxidizing agents (i.e., hydrogen peroxide) are generally mixed together in equal parts by weight (i.e., in a weight ratio of 1:1). However, weight ratios of 1:4 to 4:1 (alkalizing agent component:oxidizing agent component), preferably 1:3 to 3:1, and more preferably 1:2 to 2:1, may be selected. Use of the two components in a weight ratio of 1:1 is very particularly preferred.

If active substances or ingredients are to be used in the oxidative color-changing agent which are not compatible with either alkalizing agents or oxidizing agents, provision in the form of a three-component agent is also conceivable for increasing the storage stability. In this case, the ready-to-use agent is prepared immediately before application by the user by mixing an alkalizing agent component (I) (which optionally also includes oxidation dye precursors), an oxidizing agent component (II), and the further, third component (Ill). In each of the described cases, the ready-to-use agent is understood to mean the agent which has been obtained immediately before application by mixing all necessary individual components, and which includes all essential components for the lightening or dyeing process (i.e., alkalizing agent, oxidizing agent, and optionally oxidation dye precursors). All stated quantities refer in each case to the total weight of the ready-to-use agent.

Sodium chloride (a) and potassium chloride (b) are stable with respect to the alkalizing agents and the oxidizing agents. The mineral salts may therefore be incorporated into the component including the alkalizing agent, and also into the component including the oxidizing agent. In both cases, the formulations are characterized by excellent storage stability which is far superior to the storage stability of corresponding formulations with organic conditioner and care substances.

In summary, another preferred embodiment is characterized in that the ready-to-use oxidative color-changing agent is produced by mixing the two components (I) and (II), wherein

(I) the first component includes at least one alkalizing agent, optionally oxidation dye precursors, sodium chloride (a), and potassium chloride (b), and
(II) the second component includes hydrogen peroxide.

The mineral salts according to the invention also have excellent stability with respect to oxidizing agents, so that it is likewise possible to package the salts together with the oxidizing agent.

A likewise preferred embodiment is therefore characterized in that the ready-to-use color-changing agent is produced by mixing the two components (I) and (II), wherein (I) the first component includes at least one alkalizing agent and optionally oxidation dye precursors and

(II) the second component includes hydrogen peroxide, sodium chloride (a), and potassium chloride (b).

The optionally included salts from group (c) may be included in component (I) and/or in component (II).

As already described above, the oxidative color-changing agents including sodium chloride (a) in specific quantity ranges are particularly suitable for avoiding oxidative degradation of the peptide structure of the keratin fibers.

For this reason, a particularly preferred ready-to-use agent is characterized in that it includes sodium chloride (a) in a quantity of 0.05 to 3.5% by weight, preferably 0.1 to 2.5% by weight, more preferably 0.2 to 1.5% by weight, and particularly preferably 0.3 to 0.8% by weight, based on the total weight of the ready-to-use agent.

In an explicitly very particularly preferred embodiment, the agents according to the invention include sodium chloride and potassium chloride in specific weight ratios.

For this reason, a very particularly preferred agent according to the invention is characterized in that it includes sodium chloride (a) and potassium chloride (b) in a weight ratio (a)/(b) of 20:1 to 1:1, preferably 18:1 to 3:1, more preferably 16:1 to 6:1, and particularly preferably 14:1 to 9:1, based on the total weight of the ready-to-use agent.

For example, one preferred ready-to-use agent includes, based on the total weight of the ready-to-use agent,

(a) 0.05 to 3.5% by weight sodium chloride
(b) potassium chloride and
(d) hydrogen peroxide,
wherein sodium chloride (a) and potassium chloride (b) are included in a weight ratio (a)/(b) of 20:1 to 1:1.

Another particularly preferred ready-to-use agent includes, based on the total weight of the ready-to-use agent,

(a) 0.05 to 3.5% by weight sodium chloride
(b) potassium chloride and
(d) hydrogen peroxide,
wherein sodium chloride (a) and potassium chloride (b) are included in a weight ratio (a)/(b) of 18:1 to 3:1.

Another particularly preferred ready-to-use agent includes, based on the total weight of the ready-to-use agent,

(a) 0.05 to 3.5% by weight sodium chloride
(b) potassium chloride and
(d) hydrogen peroxide,
wherein sodium chloride (a) and potassium chloride (b) are included in a weight ratio (a)/(b) of 16:1 to 6:1.

Another particularly preferred ready-to-use agent includes, based on the total weight of the ready-to-use agent,

(a) 0.05 to 3.5% by weight sodium chloride
(b) potassium chloride and
(d) hydrogen peroxide,
wherein sodium chloride (a) and potassium chloride (b) are included in a weight ratio (a)/(b) of 14:1 to 9:1.

Normally, the user must repeat the dyeing or bleaching process at regular time intervals. At the latest when the hair has grown out and color differences are visible in the application area of the hair, a supplemental treatment must be carried out in which generally also the lengths of hair which have already undergone a color change must be retreated. If the user desires particularly intense lightening of the hair, he/she may also repeatedly treat all of the hair with the oxidative color-changing agent within short time intervals.

It has been found that the oxidative changes in the keratinic fibers may be minimized, even for repeated applications within short time intervals, when sodium chloride (a) and hydrogen peroxide (d) are used in a mutually coordinated weight ratio.

Another very particularly preferred agent according to the invention is therefore characterized in that it includes sodium chloride (a) and hydrogen peroxide (d) in a weight ratio (a)/(d) of 2:1 to 1:6, preferably 1:1 to 1:5, more preferably 1:2 to 1:4, and particularly preferably 1:3, based on the total weight of the ready-to-use agent.

The presence of further mineral salts has also been found to be advantageous with regard to the least possible oxidative change in the keratinic peptide chains.

An agent which is likewise particularly preferred according to the invention is therefore characterized in that it additionally includes at least one combination of two different salts of group (c), which is selected from calcium chloride/magnesium carbonate, calcium chloride/magnesium hydrogen carbonate, calcium chloride/magnesium sulfate, calcium chloride/magnesium hydrogen sulfate, calcium chloride/magnesium chloride, calcium chloride/calcium carbonate, calcium chloride/calcium hydrogen carbonate, calcium chloride/calcium hydrogen sulfate, and/or calcium chloride/calcium sulfate.

For example, one preferred ready-to-use agent includes

(a) sodium chloride
(b) potassium chloride
(c) magnesium carbonate and
(d) hydrogen peroxide.

Another preferred ready-to-use agent includes

(a) sodium chloride
(b) potassium chloride
(c) magnesium hydrogen carbonate and
(d) hydrogen peroxide.

Another preferred ready-to-use agent includes

(a) sodium chloride
(b) potassium chloride
(c) magnesium sulfate and
(d) hydrogen peroxide.

Another preferred ready-to-use agent includes

(a) sodium chloride
(b) potassium chloride
(c) magnesium hydrogen sulfate and
(d) hydrogen peroxide.

Another preferred ready-to-use agent includes

(a) sodium chloride
(b) potassium chloride
(c) magnesium chloride and
(d) hydrogen peroxide.

One preferred ready-to-use agent includes

(e) sodium chloride
(f) potassium chloride
(g) calcium carbonate and
(h) hydrogen peroxide.

One preferred ready-to-use agent includes

a. sodium chloride
b. potassium chloride
c. calcium hydrogen carbonate and
d. hydrogen peroxide.

One preferred ready-to-use agent includes

a. sodium chloride
b. potassium chloride
c. calcium hydrogen sulfate and
d. hydrogen peroxide.

One preferred ready-to-use agent includes

a. sodium chloride
b. potassium chloride
c. calcium sulfate and
d. hydrogen peroxide.

One preferred ready-to-use agent includes

a. sodium chloride
b. potassium chloride
c. calcium chloride and
d. hydrogen peroxide.

Another preferred ready-to-use agent includes

a. sodium chloride
b. potassium chloride
c. calcium chloride/magnesium carbonate and
d. hydrogen peroxide.

Another preferred ready-to-use agent includes

a. sodium chloride
b. potassium chloride
c. calcium chloride/magnesium hydrogen carbonate and
d. hydrogen peroxide.

Another preferred ready-to-use agent includes

a. sodium chloride
b. potassium chloride
c. calcium chloride/magnesium sulfate and
d. hydrogen peroxide.

Another preferred ready-to-use agent includes

a. sodium chloride
b. potassium chloride
c. calcium chloride/magnesium hydrogen sulfate and
d. hydrogen peroxide.

Another preferred ready-to-use agent includes

a. sodium chloride
b. potassium chloride
c. calcium chloride/magnesium chloride and
d. hydrogen peroxide.

Another preferred ready-to-use agent includes

a. sodium chloride
b. potassium chloride
c. calcium chloride/calcium carbonate and
d. hydrogen peroxide.

Another preferred ready-to-use agent includes

a. sodium chloride
b. potassium chloride
c. calcium chloride/calcium hydrogen carbonate and
d. hydrogen peroxide.

Another preferred ready-to-use agent includes

a. sodium chloride
b. potassium chloride
c. calcium chloride/calcium hydrogen sulfate and
d. hydrogen peroxide.

Another preferred ready-to-use agent includes

a. sodium chloride
b. potassium chloride
c. calcium chloride/calcium sulfate and
d. hydrogen peroxide.

The ready-to-use oxidative color-changing agents may also include additional active substances, auxiliary substances, and additives in order to enhance the dyeing and lightening power and to set further desired properties of the agents.

The ready-to-use coloring agents are preferably provided as a liquid preparation, and optionally a further surface-active substance is additionally added to the agents; depending on the field of application, such surface-active substances are referred to as surfactants or as emulsifiers. They are preferably selected from anionic, zwitterionic, amphoteric, and nonionic surfactants and emulsifiers.

Agents which are suitable according to the invention are characterized in that the agent additionally includes at least one anionic surfactant. Preferred anionic surfactants are fatty acids, alkyl sulfates, alkyl ether sulfates, and ether carboxylic acids including 10 to 20 C atoms in the alkyl group and up to 16 glycol ether groups in the molecule.

Agents which are suitable according to the invention are characterized in that the agent additionally includes at least one zwitterionic surfactant. Preferred zwitterionic surfactants are betaines, N-alkyl-N,N-dimethylammonium glycinates, N-acylaminopropyl-N,N-dimethylammonium glycinates, and 2-alkyl-3-carboxymethyl-3-hydroxyethyl imidazolines. One preferred zwitterionic surfactant is known under the INCI name Cocamidopropyl Betaine.

Agents which are suitable according to the invention are characterized in that the agent additionally includes at least one amphoteric surfactant. Preferred amphoteric surfactants are N-alkylglycines, N-alkylpropionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids, and alkylaminoacetic acids. Particularly preferred amphoteric surfactants are N-cocoalkylamino propionate, cocoacylaminoethylamino propionate, and C₁₂-C₁₈ acyl sarcosine.

In addition, it has proven to be advantageous when the agents include further, noniogenic surface-active substances. Preferred nonionic surfactants are alkyl polyglycosides and alkylene oxide addition products with fatty alcohols and fatty acids in each case including 2 to 30 mol ethylene oxide per mol fatty alcohol or fatty acid. Preparations having excellent properties are likewise obtained when they include fatty acid esters of ethoxylated glycerin as nonionic surfactants.

The nonionic, zwitterionic, or amphoteric surfactants are used in proportions of 0.1 to 45% by weight, preferably 1 to 30% by weight, and very particularly preferably 1 to 15% by weight, based on the total quantity of the ready-to-use agents.

The ready-to-use color-changing agent may also include at least one thickener. In principle, there are no limitations with regard to these thickeners. Organic as well as strictly inorganic thickeners may be used.

Suitable thickeners are anionic synthetic polymers, cationic synthetic polymers, naturally occurring thickeners, such as nonionic guar gums, scleroglucan gums, or xanthan gums, gum arabic, gum ghatti, karaya gum, gum tragacanth, carrageenan gum, agar-agar, locust bean gum, pectins, alginates, starch fractions, and derivatives such as amylose, amylopectin, and dextrins, and cellulose derivatives such as methylcellulose, carboxyalkyl celluloses, and hydroxyalkyl celluloses, nonionic synthetic polymers such as polyvinyl alcohol or polyvinylpyrrolidinone, and inorganic thickeners, in particular phyllo silicates such as bentonite, in particular smectites such as montmorillonite or hectorite.

Dyeing processes on keratin fibers customarily take place in an alkaline environment. However, to protect the keratin fibers and also the skin to the greatest extent possible, setting an excessively high pH is not desirable. It is therefore preferred when the pH of the ready-to-use agent is between 6 and 11, in particular between 8 and 10.5. pH values in the sense of the present invention are pH values that have been measured at a temperature of 22° C.

The alkalizing agents that are usable according to the invention for setting the preferred pH may be selected from the group comprising ammonia, alkanolamines, basic amino acids, and inorganic alkalizing agents such as alkaline earth or alkali metal hydroxides, alkaline earth or alkali metal metasilicates, alkaline earth or alkali metal phosphates, and alkaline earth or alkali metal hydrogen phosphates. Preferred inorganic alkalizing agents are sodium hydroxide, potassium hydroxide, sodium silicate, and sodium metasilicate. Organic alkalizing agents that are usable according to the invention are preferably selected from monoethanolamine, 2-amino-2-methylpropanol, and triethanolamine. The basic amino acids that are usable as alkalizing agents according to the invention are preferably selected from the group comprising arginine, lysine, ornithine, and histidine, particularly preferably arginine. However, within the scope of studies for the present invention, it has been found that further agents preferred according to the invention are characterized in that they additionally include an organic alkalizing agent. One embodiment of the first subject matter of the invention is characterized in that the agent additionally includes at least one alkalizing agent which is selected from the group comprising ammonia, alkanolamines, and basic amino acids, in particular ammonia, monoethanolamine, and arginine or the acceptable salts thereof.

Furthermore, it has proven to be advantageous when the coloring agents, in particular when they additionally include hydrogen peroxide, include at least one stabilizer or complexing agent. Particularly preferred stabilizers are phenacetin, alkali benzoates (sodium benzoate), and salicylic acid. In addition, all complexing agents of the prior art may be used. Complexing agents preferred according to the invention are nitrogen-including polycarboxylic acids, in particular EDTA and EDDS, and phosphonates, in particular 1-hydroxyethane-1,1-diphosphonate (HEDP) and/or ethylenediamine tetramethylene phosphonate (EDTMP) and/or diethylenetriamine pentamethylene phosphonate (DTPMP) or sodium salts thereof.

In addition, the agents according to the invention may include further active substances, auxiliary substances, and additives, for example nonionic polymers such as vinylpyrrolidinone/vinyl acrylate copolymers, polyvinylpyrrolidinone, vinylpyrrolidinone/vinyl acetate copolymers, polyethylene glycols and polysiloxanes; additional silicones such as volatile or nonvolatile, straight-chain, branched, or cyclic, crosslinked or noncrosslinked polyalkylsiloxanes (such as dimethicones or cyclomethicones), polyarylsiloxanes and/or polyalkylarylsiloxanes, in particular polysiloxanes having organofunctional groups, such as substituted or unsubstituted amines (amodimethicones), carboxyl, alkoxy, and/or hydroxyl groups (dimethicone copolyols), linear polysiloxane (A)-polyoxyalkylene (B) block copolymers, grafted silicone polymers; cationic polymers such as quaternized cellulose ethers, polysiloxanes having quaternary groups, dimethyldiallyl ammonium chloride polymers, acrylamide dimethyldiallyl ammonium chloride copolymers, dimethylamino ethyl methacrylate-vinylpyrrolidinone copolymers quaternized with diethyl sulfate, vinylpyrrolidinone-imidazolinium methochloride copolymers, and quaternized polyvinyl alcohol; zwitterionic and amphoteric polymers; anionic polymers, for example polyacrylic acids or crosslinked polyacrylic acids; structurizers such as glucose, maleic acid, and lactic acid, hair conditioning compounds such as phospholipids, for example lecithin and cephalins; fragrance oils, dimethyl isosorbide, and cyclodextrins; fiber structure-improving active substances, in particular mono-, di-, and oligosaccharides such as glucose, galactose, fructose, fruit sugar, and lactose; dyes for coloring the agent; anti-dandruff active substances such as piroctone olamines, zinc omadines, and climbazole; amino acids and oligopeptides; animal- and/or plant-based protein hydrolysates, and in the form of their fatty acid condensation products or optionally anionically or cationically modified derivatives; plant oils; light protection agents and UV blockers; active substances such as panthenol, pantothenic acid, pantolactone, allantoin, pyrrolidinone carboxylic acids and salts thereof, and bisabolol; polyphenols, in particular hydroxycinnamic acids, 6,7-dihydroxycoumarin, hydroxybenzoic acids, catechins, tannins, leucoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling agents and penetration agents such as glycerin, propylene glycol monoethyl ether, carbonates, hydrogen carbonates, guanidines, ureas, and primary, secondary, and tertiary phosphates; opacifiers such as latex, styrene/PVP and styrene/acrylamide copolymers; pearlescence agents such as ethylene glycol mono- and distearate and PEG-3-distearate; pigments, and propellants such as propane-butane mixtures, N₂O, dimethyl ether, CO₂, and air.

Those skilled in the art will select these further substances according to the desired properties of the agents. With regard to further optional components and the quantities of these components used, explicit reference is made to relevant handbooks known to those skilled in the art. The additional active substances and auxiliary substances are preferably used in the agents according to the invention in each case in quantities of 0.0001 to 25% by weight, in particular 0.0005 to 15% by weight, based on the total weight of the application mixture.

With regard to further preferred embodiments of the ready-to-use agent according to the invention, the statements concerning the use according to the invention apply mutatis mutandis. The above-described agents according to the invention have an advantageous effect on the mechanical properties and the tear strength of the treated keratin fibers, in particular with multiple applications.

A further subject matter of the present invention therefore relates to a method for the repeated, hair-protective oxidative dyeing and/or the repeated, hair-protective oxidative lightening of keratinic fibers, comprising the following steps:

(A) applying a previously described agent according to the invention to the fibers,
(B) allowing the agent applied in step (A) to act on the fibers for a period of 5 to 45 minutes, then
(C) rinsing the agent from the fibers,
wherein the method comprising steps (A) to (C) is carried out repeatedly, i.e., at least two times, preferably at least three times, on the keratinic fibers within a period of 8 weeks.

With regard to further preferred embodiments of the method according to the invention, the statements concerning the use according to the invention and the agents according to the invention apply mutatis mutandis. The cysteic acid content of keratin fibers or hair strands may be determined by near infrared (NIR) spectroscopy. NIR spectroscopy allows direct quantification of the cysteic acid content without changing the structure of the hair or destroying it (see also Y. Miyamae et al., IFSCC Magazine, 9, 219 (2006) and Y. Miyamae et al., Appl. Spectroscopy, 61, (2) 212 (2007)). In the process, the hair is irradiated with infrared radiation (thermal radiation in the near infrared range). In this way, not only is the surface of the hair analyzed, but also the NIR radiation penetrates the hair due to the small absorption cross section. The radiation excites the components of the hair, causing them to vibrate, and the radiation of defined wavelengths is absorbed. Cysteic acid results in characteristic absorptions in the NIR spectrum. The content of cysteic acid in the keratin fiber may be determined based on the intensity of these absorptions.

### Examples

Production of the Coloring Agents

The following color creams were produced (all quantities are in % by weight):

| | | E (according to |
|---|---|---|
| Color cream | V (comparison) | the invention) |
| Carbomer | 1.00 | 1.00 |
| Vitamin C | 0.20 | 0.20 |
| Cetearyl alcohol (C₁₆/C₁₈ fatty alcohol) | 2.00 | 2.00 |
| PEG-40 castor oil | 0.38 | 0.38 |
| Sodium cetearyl sulfate | 0.125 | 0.125 |
| Disodium cocoamphodipropionate | 4.20 | 4.20 |
| Monoethanolamine | 5.00 | 5.00 |
| Xiameter OFX-0193 Fluid | 1.00 | 1.00 |
| (INCI: PEG-12 Dimethicone) | | |
| Sodium sulfite | 0.20 | 0.20 |
| p-Toluylenediamine, sulfate | 3.26 | 3.26 |
| Resorcinol | 0.65 | 0.65 |
| 4-Chlororesorcinol | 0.71 | 0.71 |
| m-Aminophenol | 0.15 | 0.15 |
| p-Amino-o-cresol (5-amino-2- | 0.08 | 0.08 |
| methylphenol) | | |
| 3-Amino-2-methylamino-6- | 0.22 | 0.22 |
| methoxypyridine | | |
| 1-Hydroxyethane-1,1-diphosphonic acid | 0.12 | 0.12 |
| Taurine | 2.00 | — |
| Caffeine | 0.50 | — |
| La Toja mineral salts* | — | 1.00 |
| Fragrance | 0.60 | 0.60 |
| Water | ad 100 | ad 100 |

*Composition of “La Toja” mineral salt mixture: Sodium chloride: 78.2% by weight Potassium chloride: 7.01% by weight Calcium chloride: 8.40% by weight Magnesium chloride: 1.64% by weight Calcium sulfate: 1.59% by weight Further salts: ad 100% by weight

The color creams V and E were each mixed in a 1:1 weight ratio with the following oxidizing agent preparation:

| Oxidizing agent preparation | |
|---|---|
| EDTA (ethylenediamine tetraacetic | 0.15 |
| acid), disodium salt | |
| Disodium pyrophosphate | 0.30 |
| Sodium benzoate | 0.04 |
| Cetearyl alcohol | 1.68 |
| PEG-40 castor oil | 0.32 |
| Sodium cetearyl sulfate | 0.10 |
| Hydrogen peroxide | 6.00 |
| (50% aqueous solution) | |
| Phosphoric acid | 0.04 |
| Water | ad 100 |

2. Application

The ready-to-use oxidative coloring agents produced in this way were each applied to five hair strands (Kerling, Euronaturhaar, 6-0), allowed to act for 10 minutes, and rinsed off. The hair strands were subsequently dried. The application was repeated three times on each hair strand.

3. Measurement of the Cysteic Acid Content

To measure the damage to hair caused by the triple application, the cysteic acid value of each treated hair strand was determined by quantitative NIR spectroscopy. The spectra were recorded using an MPA™ FT-NIR spectrometer from Bruker Optic GmbH. The infrared range includes the wave number range from 12,500 cm⁻¹ to 4000 cm⁻¹, and is characteristic for overtone and combination vibrations of CH, OH, and NH groups, for example.

The measurement of the samples was carried out using the integrated sphere module at six different sample positions in diffuse reflection. The wave number range of 7300 cm⁻¹ to 4020 cm⁻¹ was selected for the analysis of the measured NIR spectra.

The NIR spectra of cystine show characteristic absorption bands in the wave number range of 6200 cm⁻¹ to 5500 cm⁻¹. If the hair changes due to fairly severe damage (i.e., the cysteic acid content in the hair increases), in the NIR spectrum this has an effect on the bands at 5020 cm′ to 4020 cm′, which are characteristic for cysteic acid. The NIR spectra were quantitatively evaluated by computer.

NIR analysis value [mol cysteic acid/100 mol amino acid]

| Application | Hair | Hair | Hair | Hair | Hair | Average |
|---|---|---|---|---|---|---|
| mixture | strand 1 | strand 2 | strand 3 | strand 4 | strand 5 | value |
| Untreated | 0.8 | 1.0 | 0.7 | 0.9 | 0.7 | 0.8 |
| V | 1.3 | 1.1 | 1.3 | 1.1 | 1.1 | 1.2 |
| dyed 3 x | | | | | | |
| E | 0.9 | 1.1 | 1.2 | 0.9 | 0.9 | 1.0 |
| dyed 3 x | | | | | | |

Hair strands which were treated three times with the oxidative coloring agent (E) according to the invention showed a significantly reduced cysteic acid value in comparison to the comparative formulation (V).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method, comprising
applying to keratinic fibers a ready-to-use agent for oxidatively dyeing and/or oxidatively lightening keratinic fibers while reducing damage to the keratinic fibers resulting from use of the agent, the agent including
(a) sodium chloride.

2. The method according to claim 1, wherein the agent additionally includes
(b) potassium chloride.

3. The method according to claim 1, wherein the agent includes the sodium chloride (a) in a quantity of 0.05 to 3.5% by weight based on the total weight of the ready-to-use agent.

4. The method according to claim 2, wherein the agent includes the potassium chloride (b) in a quantity of 0.01 to 1.0% by weight based on the total weight of the ready-to-use agent.

5. The method according to claim 2, wherein the agent includes the sodium chloride (a) and the potassium chloride (b) in a weight ratio (a)/(b) of 20:1 to 1:1 based on the total weight of the ready-to-use agent.

6. The method according to claim 2, wherein the agent additionally includes at least one further salt (c) selected from the group consisting of magnesium carbonate, magnesium hydrogen carbonate, magnesium sulfate, magnesium hydrogen sulfate, magnesium chloride, calcium carbonate, calcium hydrogen carbonate, calcium hydrogen sulfate, calcium sulfate, and calcium chloride.

7. The method according to claim 6, wherein the agent includes at least one combination of two different salts of group (c) selected from the group consisting of calcium chloride/magnesium carbonate, calcium chloride/magnesium hydrogen carbonate, calcium chloride/magnesium sulfate, calcium chloride/magnesium hydrogen sulfate, calcium chloride/magnesium chloride, calcium chloride/calcium carbonate, calcium chloride/calcium hydrogen carbonate, calcium chloride/calcium hydrogen sulfate, and calcium chloride/calcium sulfate.

8. The method according to claim 1, wherein the agent further includes hydrogen peroxide as an oxidizing agent (d).

9. The method according to claim 8, wherein the agent includes the sodium chloride (a) and the hydrogen peroxide (d) in a weight ratio (a)/(d) of 2:1 to 1:10 based on the total weight of the ready-to-use agent.

10. A ready-to-use agent for oxidatively dyeing and/or oxidatively lightening keratinic fibers, including
(a) sodium chloride,
(b) potassium chloride,
(c) optionally at least one further salt selected from the group consisting of magnesium carbonate, magnesium hydrogen carbonate, magnesium sulfate, magnesium hydrogen sulfate, magnesium chloride, calcium carbonate, calcium hydrogen carbonate, calcium hydrogen sulfate, calcium sulfate, and calcium chloride, and
(d) hydrogen peroxide.

11. The agent according to claim 10, wherein the agent includes the sodium chloride (a) in a quantity of 0.05 to 3.5% by weight based on the total weight of the ready-to-use agent.

12. The agent according to claim 10, wherein the agent includes the sodium chloride (a) and the potassium chloride (b) in a weight ratio (a)/(b) of 20:1 to 1:1 based on the total weight of the ready-to-use agent.

13. The agent according to claim 10, wherein the agent includes the sodium chloride (a) and the hydrogen peroxide (d) in a weight ratio (a)/(d) of 2:1 to 1:6 based on the total weight of the ready-to-use agent.

14. The agent according to claim 10, wherein the agent includes at least one combination of two different salts of group (c), which is selected from the group consisting of calcium chloride/magnesium carbonate, calcium chloride/magnesium hydrogen carbonate, calcium chloride/magnesium sulfate, calcium chloride/magnesium hydrogen sulfate, calcium chloride/magnesium chloride, calcium chloride/calcium carbonate, calcium chloride/calcium hydrogen carbonate, calcium chloride/calcium hydrogen sulfate, and calcium chloride/calcium sulfate.

15. A method for the repeated, hair-protective oxidative dyeing and/or the repeated, hair-protective oxidative lightening of keratinic fibers, which includes:
(A) applying an agent according to claim 10 to the fibers,
(B) allowing the agent applied in step (A) to act on the fibers for a period of 5 to 45 minutes, and then
(C) rinsing the agent from the fibers,
wherein steps (A) to (C) are carried out at least two times on the keratinic fibers within a period of 8 weeks.
